(19) United States
(12) Patent Application Publication
(10) Pub. No.: US 2022/0211978 A1
(43) Pub. Date: Jul. 7, 2022

(54) HOLDING MECHANISM ASSEMBLY

(71) Applicant: HI-LEX CORPORATION, Takarazuka-shi, Hyogo (JP)

(72) Inventor: Shinsaku Koarashi, Takarazuka-shi, Hyogo (JP)

(21) Appl. No.: 17/607,135

(22) PCT Filed: May 7, 2020
(86) PCT No.: PCT/JP2020/018485

§ 371 Date: Oct. 28, 2021

(30) Foreign Application Priority Data
May 10, 2019 (JP) 2019-089787

(51) Int. Cl. A61M 25/02; A61M 39/02; A61M 60/122; F16J 15/06

(52) U.S. Cl. CPC A61M 25/02; A61M 39/02; A61M 2025/028; F16J 15/06; A61M 60/122

(57) ABSTRACT

A holding mechanism assembly 1 includes: a tubular inserting member 2; a fixing device 3 having a fixing portion 31 and a communicating portion 32; and a holding structure 4 to hold the inserting member 2 to the fixing device 3 in a liquid tight manner. The communicating portion 32 has a first opening 321 opened toward the outside of the living body; a second opening 322 opened toward the inside of the living body; and a communication path 323 formed between the first opening 321 and the second opening 322. An outer surface 2a of the inserting member 2 has a covered region A1 covered with fiber that can guide cells and a non-covered region A2 not covered with fiber, and the non-covered region A2 extends between the holding structure 4 and the second opening 322.

### TECHNICAL FIELD

The invention relates to a holding mechanism assembly.

### BACKGROUND ART

A tubular body (for example, a medical tube), which is to be inserted through the living body and communicates the inside and the outside of the living body, is held to the living body by a holding mechanism assembly for holding the tubular body to an insertion target. (see Patent document 1, for example). The holding mechanism assembly of Patent document 1 comprises: a driveline being a medical tube; a fixing device having an inserting portion into which the driveline is inserted; and a holding structure being provided in the inserting portion to hold the driveline in a liquidtight manner. The driveline is inserted into the holding structure provided in the inserting portion passing through the fixing device and is held by the fixing device. To fix the driveline to the living body, the fixing device is fixed to the incised area of the skin of the living body.

The holding structure provided in the inserting portion of the fixing device of Patent document 1 is provided in a part of the inserting portion in the axial direction of the inserting portion, not provided across the entirety in the axial direction of the inserting portion. Thus, a space is formed, in the radial direction of the driveline, between the inner surface of the inserting portion and the outer surface of the driveline extending by passing through the holding structure. When a predetermined time period elapses after the fixing device is fixed to the skin, cells surrounding the site to which the fixing device is fixed reproduce, so that the space formed between the inner surface of the inserting portion and the outer surface of the driveline is filled up with the reproduced cells.

### PRIOR ART DOCUMENT

### Patent Document

Patent Document 1: JP 2017-104437 A

### SUMMARY OF THE INVENTION

### Problem to be Solved by the Invention

However, in the above-described holding mechanism assembly, when the living body performs various actions after the holding mechanism assembly is attached to the living body, a relative movement between the driveline and the fixing device may occur. When such a relative movement occurs, cells invading into the above-mentioned space may be damaged to cause inflammation. In order to suppress such inflammation, predetermined measures against inflammation, for example, medication to suppress propagation of bacteria, needs to be taken.

An object of the invention is to provide a holding mechanism assembly that makes it possible to suppress cells being damaged in a space formed between an outer surface of a driveline and an inner wall of a communicating portion into which an inserting member is inserted.

### Means to Solve the Problem

A holding mechanism assembly according to the invention comprises: a tubular inserting member, one end side of which is to be arranged in an inside of a living body and the other end side of which is to be arranged in an outside of the living body; a fixing device having a fixing portion to be fixed beneath a skin of the living body and a communicating portion into which the inserting member is inserted; and a holding structure provided in the communicating portion to hold the inserting member to the fixing device in a liquidtight manner, wherein the communicating portion has a first opening opened toward the outside of the living body when the fixing device is fixed beneath the skin; a second opening opened toward the inside of the living body when the fixing device is fixed beneath the skin; and a communication path formed between the first opening and the second opening; a space is formed, on the second opening side of the communication path with respect to the holding structure, between an inner wall of the communicating portion, which defines the communication path, and an outer surface of the inserting member; the outer surface of the inserting member has a covered region covered with fiber that can guide cells and a non-covered region not covered with the fiber; and the non-covered region of the inserting member extends between the holding structure and the second opening in the communication path.

### Effects of the Invention

The holding mechanism assembly according to the invention makes it possible to suppress cells being damaged in a space formed between an outer surface of a driveline and an inner wall of a communicating portion into which an inserting member is inserted.

### BRIEF DESCRIPTION OF THE DRAWINGS

FIG. 1 shows a lateral partial cross-sectional view of an overall configuration of a holding mechanism assembly according to one embodiment of the invention.

FIG. 2 shows an exploded perspective view of the holding mechanism assembly in FIG. 1.

FIG. 3 shows a partially enlarged cross sectional view of a holding structure of the holding mechanism assembly in FIG. 1.

FIG. 4 shows a top view of a fixing device according to one embodiment of the invention.

FIG. 5 is a schematic view showing the state of the living body at the time the holding mechanism assembly of FIG. 1 is attached to the living body.

FIG. 6 is a schematic view showing the state in which the inserting member moves relatively with respect to the fixing device in a reference example in which fiber is provided on the outer surface, which opposes the inner wall of the communicating portion of the fixing device, of the inserting member.

FIG. 7 is a schematic view showing the state in which the inserting member moves relatively with respect to the fixing device, in the holding mechanism assembly of FIG. 1.

### EMBODIMENT FOR CARRYING OUT THE INVENTION

Below, a fixing device according to one embodiment of the invention is explained with reference to the attached drawings. The embodiment shown below is merely one example, so that a fixing device in the invention is not limited to the embodiment below.

First, a holding mechanism assembly including a fixing device according to one embodiment of the invention will be explained.

The holding mechanism assembly is a structure for holding an inserting member to be inserted through an insertion target. Here, “insert” means that the inserting member having a predetermined length is at least in a state in which the inserting member is present across the interior and the exterior of the holding mechanism assembly. As long as the inserting member is inserted through the insertion target, the inserting member may pass through the insertion target or not pass through the insertion target. The insertion target has a hole through which the inserting member is to be inserted. The inserting member has a predetermined length.

For the inserting member, a flexible medical tube may be used as one example. In the embodiment, a driveline being one type of medical tube is used. The above-mentioned driveline is inserted from tissues outside the living body, such as the skin into inside the body, such as an organ. The inserting member is not limited to the driveline, so that the inserting member may be a catheter, a different medical tube, or a solid medical cable, or may be a different member having a predetermined length.

<Configuration of Holding Mechanism Assembly>

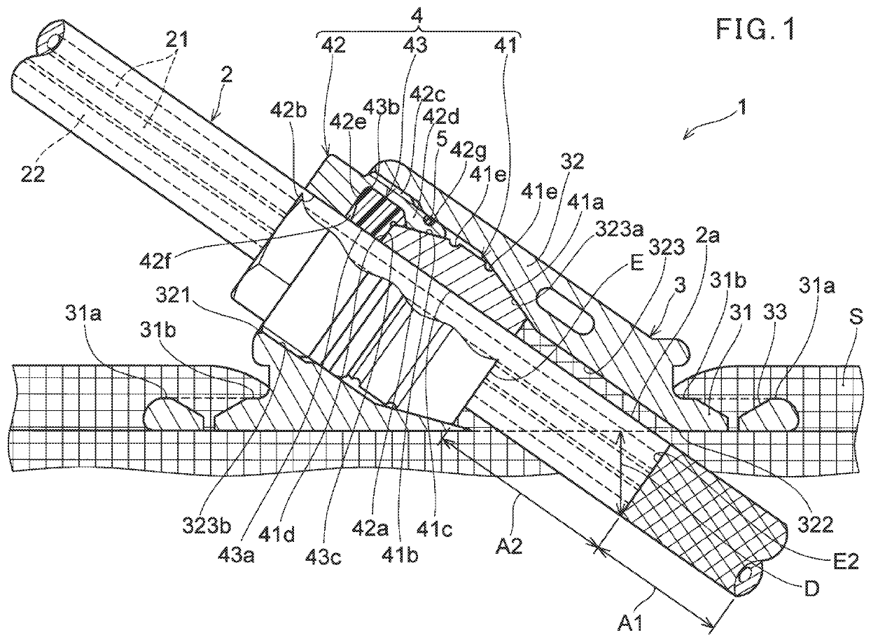

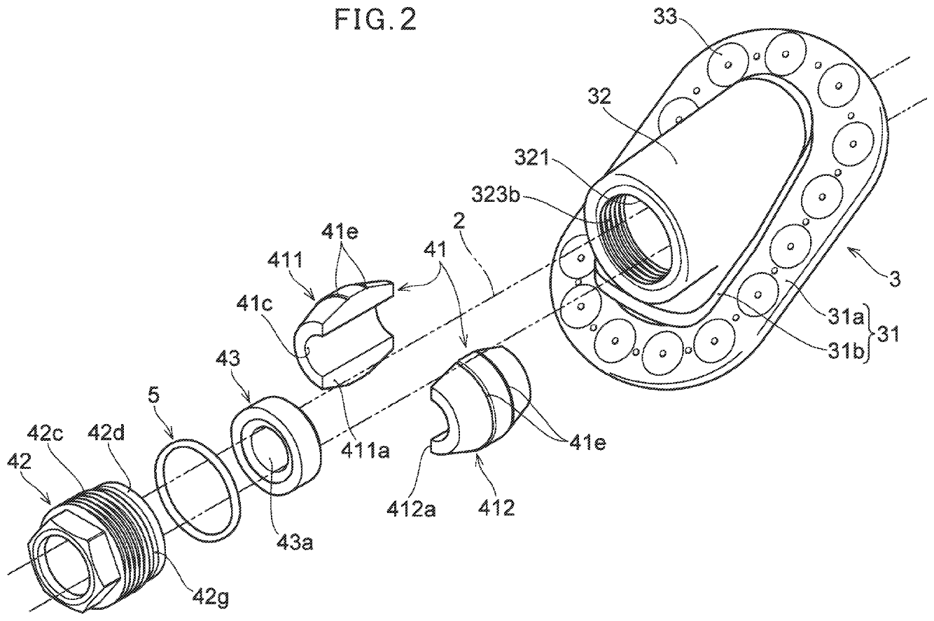

Next, the overall configuration of the holding mechanism assembly will be explained using FIGS. 1 and 2.

As shown in FIG. 1, a holding mechanism assembly 1 comprises: a tubular inserting member 2, one end side of which is to be arranged in an inside of the living body and the other end side of which is to be arranged in an outside of the living body; a fixing device 3 having a fixing portion 31 to be fixed beneath the skin of the living body and a communicating portion 32 into which the inserting member 2 is inserted; and a holding structure 4 provided in the communicating portion 32 to hold the inserting member 2 to the fixing device 3 in a liquidtight manner. Here, the fixing device 3 is fixed to the skin S by anchoring (fixing) a part of the skin S to the surface of the fixing device 3.

According to the embodiment, the inserting member 2 is a driveline as described above (below called a driveline 2). The driveline 2 is a tubular member. An end of the driveline 2 inside the body is coupled to a medical device (not shown) being arranged in the body. An end of the driveline 2 outside the body is coupled to a device (not shown) being arranged outside the body. A connector (not shown) for coupling a device (not shown) may be mounted to the end of the driveline 2 outside the body. The outer diameter of the connector may be greater or less than the outer diameter of the driveline 2.

In the embodiment, the medical device arranged inside the body is an auxiliary artificial heart. The auxiliary artificial heart is a member having a pump to circulate blood in the body. The device arranged outside the body includes a pressure-feeding pump to send out cooling water to cool the above-mentioned pump and a power source to feed electric power to the pump. The driveline 2 has, in the interior thereof, a cooling water circulation path 21 to circulate cooling water between the pressure-feeding pump and the auxiliary artificial heart, and a power cable 22 to connect the power source and the pump. The configuration of the driveline 2 is not limited thereto, so that a publicly-known configuration may be adopted. For example, the driveline 2 may be configured to have only a power cable, may be configured to have only a cooling water circulation path, or may be configured to have a member having a different function. The driveline 2 includes the cooling water circulation path 21 containing water inside thereof and the power cable 22, and is used as a medical tube having flexibility and rigidity.

The fixing device 3 has a fixing portion 31 to be fixed to the skin S and a communicating portion 32 through which the driveline 2 is inserted. A surface treatment is applied to at least a part of the fixing device 3 to promote anchoring of a part of the skin S. A material for the fixing device 3 is not particularly limited as long as the fixing device 3 has rigidity with which the fixing device 3 can be fixed to a target. In a case that the fixing device 3 is a member to be fixed to the skin S being biological tissues as in the embodiment, it is preferable that the fixing device 3 is formed of a metal having a high biocompatibility. Metals having the high biocompatibility include titanium and a titanium alloy, for example. The surface of the fixing portion 31 is formed with a porous material such as a mesh-shaped titanium fiber, into which porous material cells can invade, improving close contactability between the fixing portion 31 and the skin S to achieve the prevention of invasion of bacteria into the living body.

The fixing portion 31 of the fixing device 3 is fixed to the skin S by inducing the skin tissues of the incised skin S to the fixing portion 31. In the embodiment, as shown in FIGS. 1, 2, and 4, the fixing portion 31 is extended from the communicating portion 32 outwardly in the radial direction of the communicating portion 32 at the end on the living body-side of the communicating portion 32. In the embodiment, the fixing portion 31 is formed in a flange shape to surround the communicating portion 32. The fixing portion 31 is formed in a plate shape provided with the communication path 323 of the communicating portion 32 passing therethrough. As shown in FIGS. 1 and 2, in the fixing portion 31, a plurality of countersink portions 33 each being a concave portion are provided on a first surface on the first opening 321 side. In the countersink portion 33, the skin tissues can enter the space of the concave portion, which countersink portion 33 is provided in a grinding bowl shape.

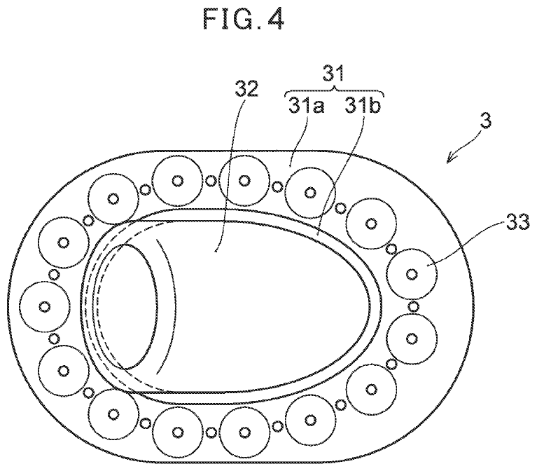

The fixing portion 31 of the fixing device 3, as shown in FIG. 4, has a porous portion 31a capable of inducing cells to the space of the porous portion 31a and a flat portion 31b that allows the cells to be adhered thereto.

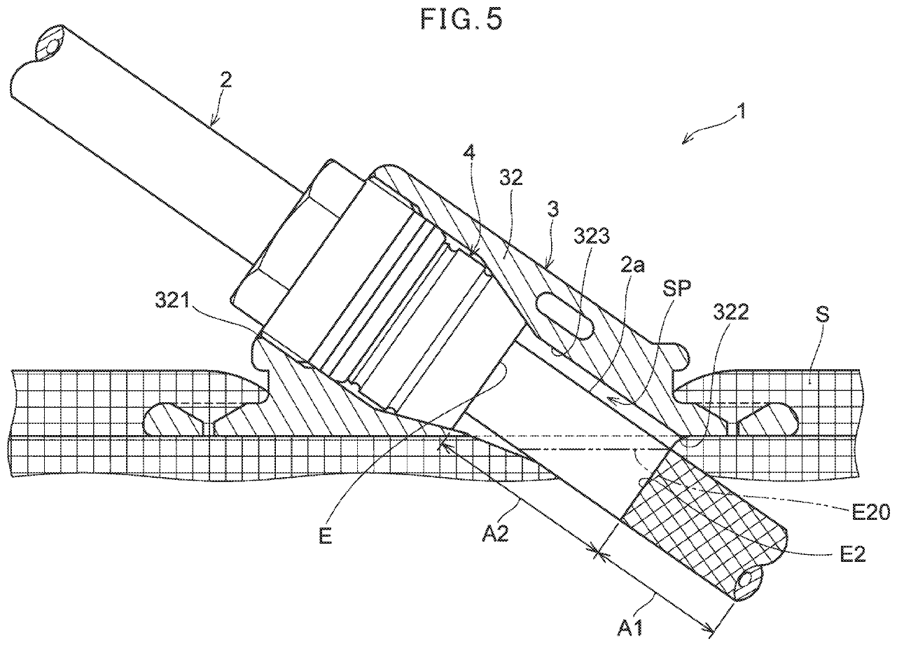

The porous portion 31a has a plurality of pores having a pore diameter to induce the cells into the pores. The porous portion 31a is, of the fixing portion 31, a part in which a large number of pores into which the cells can enter to be fixed thereto are formed. While a single pore that allows the cells to invade thereinto can be provided in a plurality in the porous portion 31a, the porous portion 31a preferably has a porous layer in which a plurality of pores are communicatively connected three-dimensionally. The skin S is fixed to the porous portion 31a by inducing the cells in the thickness direction of the porous portion 31a (in the downward direction in FIG. 5) to pores (air gaps) formed in the porous portion 31a. As long as the cells can invade and grow in the porous portion 31a, the material for the porous portion 31a or the size of the pores is not particularly limited. For the porous portion 31a, a porous material known in the medical field, such as a mesh-shaped titanium fiber may be used, for example. As the porous portion 31a is a part fixing the fixing device 3 to the skin, the porous portion 31a is preferably a hard member and is more preferably made of a metal such as titanium. Moreover, it is preferable that the porous portion 31a is provided integrally with the fixing portion 31 and it is more preferable that the porous portion 31a is provided integrally with the fixing device 3.

The flat portion 31b has a substantially flat surface and has primarily a function of causing the cells to adhere to the surface of the flat portion 31b. A material for the flat portion 31b is not particularly limited as long as the cells can adhere to the surface SF2 of the flat portion 31b. The flat portion 31b may be configured by a material having a high biocompatibility, such as titanium or a titanium alloy, for example. Furthermore, the flat portion 31b may be made to have a desired surface condition by providing the material having the high biocompatibility with a publicly-known surface treatment such as a mirror surface process or a blasting process (particularly a grit blasting process).

The communicating portion 32 communicates the inside of the living body and the outside of the living body. In the embodiment, the communicating portion 32 is a portion through which an inserting member such as the driveline 2 is inserted and is configured in a substantially cylindrical shape. The communicating portion 32 has an opening for the inside of the living body and an opening for the outside of the living body. The communicating portion 32 has the first opening 321, a second opening 322, and the communication path 323 formed between the first opening 321 and the second opening 322. Moreover, in the embodiment, the communicating portion 32 has a communicating portion inclined surface 323a provided at the inner surface of the communication path 323 such that the inner diameter is reduced from the first opening 321 toward the second opening 322, and a female screw portion 323b provided on the inner peripheral surface of the first opening 321. In each of the drawings, illustration of the screw groove of the female screw portion 323b is omitted.

The first opening 321 is an opening opened toward the outside of the living body when the fixing device 3 is fixed beneath the skin, while the second opening 322 is an opening opened toward the inside of the living body when the fixing device 3 is fixed beneath the skin. The first opening 321 is formed such that the opening surface thereof is orthogonal with respect to the axial direction of the communicating portion 32. The second opening 322 is formed such that the opening surface thereof is non-orthogonal with respect to the axial direction of the communicating portion 32. The cross section of the first opening 321 is formed in a circular shape, and the diameter of the first opening 321 in the cross section is substantially same as the outer diameter of a screw member 42 to be described below. The cross section of the second opening 322 is formed in an elliptical shape, and the cross-sectional area of the second opening 322 is formed to be greater than the cross-sectional area of the driveline 2 when the driveline 2 is cut in a direction being parallel to the second opening 322.

The communication path 323 is a path to communicate the first opening 321 and the second opening 322. The communication path 323 is formed in a circular shape in a cross sectional view. The communication path 323 is configured such that the central axis thereof forms one straight line. While the communication path 323 is formed such that the central axis thereof forms one straight line, it is not limited thereto. The communication path 323 may be formed to be curved or bent at the intermediate portion of the communication path 323, for example. Moreover, in the embodiment, the communication path 323 is configured to be inclined with respect to the fixing portion 31, but the inclined angle is not particularly limited. Furthermore, the communication path may be formed to be orthogonal to the target.

The communicating portion inclined surface 323a is a portion provided on the inner surface of the communication path 323 and is provided such that the inner diameter thereof is reduced toward the second opening 322 from the first opening 321. The communicating portion inclined surface 323a is provided such that the inclined angle of the communicating portion inclined surface 323a is constant. In other words, the communicating portion inclined surface 323a is configured in a straight-line shape in the cross section shown in FIG. 1. While the communicating portion inclined surface 323a is formed such that the inclined angle thereof is constant in the embodiment, it is not limited thereto. For example, the communicating portion inclined surface 323a may also be formed such that the inclined angle of the communicating portion inclined surface 323a increases toward the second opening 322. In this case, the communicating portion inclined surface is configured such that the cross section shown in FIG. 1 is in a curved shape.

The female screw portion 323b is a portion provided on the inner peripheral surface of the first opening 321. A male screw portion 42c to be described below screws into the female screw portion 323b.

In the embodiment, the holding structure 4 is a structure so that the fixing device 3 holds the driveline 2 in a liquid-tight manner to keep inside of the body in a liquid-tight manner. The holding structure 4 comprises a chuck member 41, a screw member 42, and a sealing member 43.

The chuck member 41 is a member to tighten and grasp an inserting member such as the driveline 2. The chuck member 41 has a fixing device-side contacting portion 41a which is provided on an outer periphery of one end side of the chuck member 41 and contacts the communicating portion inclined surface 323a, a screw member-side contacting portion 41b which is provided on an outer peripheral surface on the other end side of the chuck member 41 and contacts the screw member 42, a fitting portion 41c which is provided on the inner side and fits to the outer periphery of the driveline 2 in close contact therewith, and a sealing member-side contacting portion 41d to contact the sealing member 43. The chuck member 41 is a member to be arranged between the inner wall of the communicating portion 32 and the outer surface of the driveline 2. The chuck member 41 has, in the holding structure 4, a role of holding the driveline 2 by tightening the driveline 2 and a role of achieving liquid tightness. A material for the chuck member is not particularly limited. A material capable of being slightly deformed such as to make it possible to tighten the driveline 2 may be used as a material capable of achieving the role of holding the insertion target and the role of achieving the liquid tightness. The chuck member 41 in the embodiment is formed of a metal having a high corrosion resistance, which metal has the hardness equivalent to that of a material forming the fixing device 3 or the hardness lower than that of the material forming the fixing device 3. The metal having the high corrosion resistance may be titanium or a titanium alloy, for example.

In a state where the chuck member 41 is arranged in the communicating portion 32, the fixing device-side contacting portion 41a is a contacting portion provided at an end on the second opening 322 side and is a portion which contacts the communicating portion inclined surface 323a. The fixing device-side contacting portion 41a is configured to contact the communicating portion inclined surface 323a such that a force in a direction in which the chuck member 41 is reduced in diameter (a force toward the driveline 2) is applied to the chuck member 41 from the communicating portion inclined surface 323a. In the embodiment, the inclined angle of the fixing device-side contacting portion 41a is identical to that of the communicating portion inclined surface 323a. In other words, the fixing device-side contacting portion 41a is configured to contact the communicating portion inclined surface 323a in a surface contact with the communicating portion inclined surface 323a. In other words, an inclined surface having identical inclined angle to that of the communicating portion inclined surface 323a is preferably formed in at least one portion of the fixing device side contacting portion 41a.

The screw member-side contacting portion 41b is a contacting portion provided at an end on the first opening 321 side in a state where the chuck member 41 is arranged in the communicating portion 32 to contact the screw member 42. The screw member-side contacting portion 41b is configured as a part of an inclined surface formed on the first opening 321 side of the chuck member 41. The screw member-side contacting portion 41b is configured to contact the tip contacting portion 42a of the screw member 42 (described below) such that a force in the direction in which the chuck member 41 is reduced in diameter (a force toward the driveline 2) is applied to the chuck member 41 from the tip contacting portion 42a. Moreover, the tip contacting portion 42a of the screw member 42 also receives, from the screw member-side contacting portion 41b, a force outwardly in a radial direction of the screw member 42. In the embodiment, the screw member-side contacting portion 41b is configured by an inclined surface whose inclined angle is identical to that of the tip contacting portion 42a being an inclined surface. In other words, the screw member-side contacting portion 41b is configured to contact the tip contacting portion 42a in a surface contact with the tip contacting portion 42a. In other words, an inclined surface having inclined angle identical to that of the tip contacting portion 42a is preferably formed in at least one portion of the screw member-side contacting portion 41b.

While the screw member-side contacting portion 41b is configured with an inclined surface, it is not limited thereto. It suffices that the screw member-side contacting portion 41b has a shape capable of receiving a force from the screw member 42 in a direction in which the chuck member 41 is at least pushed toward the second opening 322 side.

The fitting portion 41c is a portion to fit to the outer periphery of the driveline 2 in close contact therewith. In the embodiment, the fitting portion 41c is the entire inner peripheral surface of the chuck member 41. In the embodiment, the chuck member 41 has a communicating hole to communicate an opening at a side of the first opening 321 and an opening at a side of the second opening 322 and grasps the driveline 2 such that the driveline 2 extends out of these openings, and the fitting portion 41c is the entire inner peripheral surface of the communicating hole of the chuck member 41.

The fitting portion 41c is formed in a circular shape in a cross section and is formed such that the diameter of the cross section of the fitting portion 41c is uniform as shown in FIG. 1. While the fitting portion 41c is formed such that the diameter of the cross section thereof is uniform, it is not limited thereto. The fitting portion 41c may have a step portion formed such that the diameter of the cross section increases in the central region of the fitting portion 41c, for example.

The sealing member-side contacting portion 41d is a contacting portion provided at an end on the first opening 321 side in a state where the chuck member 41 is arranged in the communicating portion 32 to contact the sealing member 43. The sealing member-side contacting portion 41d is configured with a part of the inclined surface formed on the first opening 321 side of the chuck member 41 and a surface being parallel to the radial direction of the communicating portion 32. The sealing member-side contacting portion 41d is continuously formed with the screw member-side contacting portion 41b. While the sealing member-side contacting portion 41d is configured with the part of the inclined surface formed on the first opening 321 side of the chuck member 41 and the surface being parallel to the radial direction of the communicating portion 32, it is not limited thereto. It suffices that the sealing member-side contacting portion 41d has a shape capable of receiving a force from the sealing member 43 in a direction in which the chuck member 41 is pushed toward the second opening 322 side.

As shown in FIG. 2, the chuck member 41 in the embodiment configured by a first chuck member 411 and a second chuck member 412. The chuck member 41 may be divided in the radial direction and may be a substantially tubular body by integrally joining them. The chuck member 41 is formed when a joining surface 411a of the first chuck member 411 and a joining surface 412a of the second chuck member 412 are joined. Moreover, the chuck member 41 has a concave portion 41e on the outer peripheral surface of the chuck member 41. The concave portion 41e is provided in order to stop sliding of the chuck member 41 when the chuck member 41 is attached to the insertion target. The structure of the chuck member 41 is not limited to the structure capable of being divided in the radial direction. The chuck member 41 may be configured as the structure capable of being divided in the axial direction or the structure not capable of being divided.

The screw member 42 is a member being screwed into the female screw portion 323b being provided on the inner peripheral surface of the first opening 321 to close the first opening 321 and hold the driveline 2. The screw member 42 has a screw member inserting path 42b through which the driveline 2 is inserted, a male screw portion 42c provided on the outer peripheral surface, a tip portion 42d to be inserted into the communicating portion 32, a tip contacting portion 42a provided on the inner side of the tip portion 42d to contact the screw member-side contacting portion 41b, and a screw member fitting portion 42e to fit to the sealing member 43. The screw member 42 is arranged between the inner wall of the communicating portion 32 and the outer peripheral surface of the sealing member 43 and between the inner peripheral surface of the communicating portion 32 and the outer peripheral surface of the chuck member 41. The screw member 42 has, in the holding structure 4, a role of closing the first opening 321, a role of pressing the chuck member 41 and the sealing member 43, and a role of holding the driveline 2. A material for the screw member is not particularly limited as long as the material is capable of achieving the role of closing the first opening 321, the role of pressing the chuck member 41 and the sealing member 43, and a role of holding an insertion target. In the embodiment, the screw member 42 is formed of a metal having a high corrosion resistance, which metal has the hardness equivalent to that of a material forming the fixing device 3 or the hardness lower than that of the material forming the fixing device 3. The metal having the high corrosion resistance may be titanium or a titanium alloy, for example.

The screw member inserting path 42b is a path provided inside the screw member 42 to insert the driveline 2 through the screw member 42. The screw member inserting path 42b is provided from an end of the screw member 42 on the outer side of the living body to the intermediate portion of the screw member 42. The diameter of the screw member inserting path 42b is substantially same as the outer diameter of the driveline 2.

The male screw portion 42c is a portion provided on the outer peripheral surface of the screw member 42 to be screwed into the female screw portion 323b.

The tip portion 42d is a portion to be inserted into the communicating portion 32 of the fixing device 3, and the male screw portion 42c is provided on the outer peripheral surface thereof. In each of the drawings, illustration of threads of the male screw portion 42c may be omitted.

The tip contacting portion 42a is a portion being provided on the inner peripheral surface of the tip portion 42d to contact the screw member-side contacting portion 41b. The tip contacting portion 42a is a portion to contact the screw member-side contacting portion 41b configuring the chuck member 41 and is configured with an inclined surface having an inclined angle being identical to the inclined angle of the inclined surface of the screw member-side contacting portion 41b. The tip contacting portion 42a is formed continuously with the screw member fitting portion 42e. While the tip contacting portion 42a is configured with an inclined surface having an inclined angle being identical to the inclined angle of the inclined surface of the screw-member side contacting portion 41b, the tip contacting portion 42a is not limited thereto. It suffices that the tip contacting portion 42a has a shape capable of transmitting a force from the screw member 42 in a direction in which the chuck member 41 is pushed toward the second opening 322 by contacting at least a part of the tip contacting portion 42a with the screw-member side contacting portion 41b.

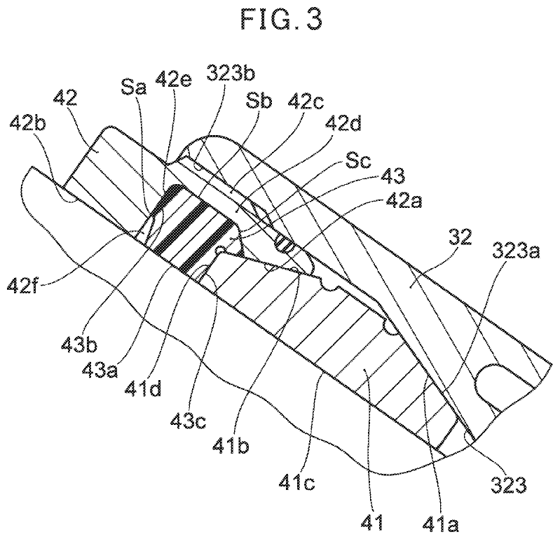

The screw member fitting portion 42e is a portion to fit to the sealing member 43. A pressing surface Sa to press the sealing member 43 in the axial direction of the driveline 2 is provided to the screw member fitting portion 42e. As shown in FIG. 3, the screw member fitting portion 42e has the pressing surface Sa (See FIG. 3) provided at the first opening 321 side and being parallel to the radial direction, a contact surface Sb being parallel to the outer peripheral surface of the sealing member 43, and a contact surface Sc being provided such that the inner diameter of the contact surface Sc is reduced toward the second opening 322 side from the first opening 321 side. The configuration of the screw member fitting portion is not limited to the configuration shown in the embodiment as long as the screw member fitting portion fits to the sealing member and has the pressing surface.

Moreover, the screw member 42 has a receiving portion 42f to receive a deformed portion of the sealing member 43 when the sealing member 43 is pressed and deformed. The receiving portion 42f is provided on the first opening 321 side of the screw member fitting portion 42e and at the inner side (the screw member inserting path 42b side) in the radial direction.

Furthermore, a concave portion 42g to arrange an O-ring 5 is provided on the outer periphery of the screw member 42. The concave portion 42g is provided on the outer peripheral surface of the tip portion 42d. The O-ring 5 is a member provided between the outer peripheral surface of the screw member 42 and the inner peripheral surface of the communicating portion 32 and has a role of assisting the liquidtightness between the screw member 42 and the communicating portion 32. The O-ring 5 may be attached to the inner peripheral surface of the communicating portion 32, not on the outer periphery of the screw member 42.

The sealing member 43 has an inserting member-side pressing portion 43a to elastically press the outer periphery of the driveline 2, a sealing member fitting portion 43b to fit to the screw member 42, and a chuck member-side contact surface 43c to contact the chuck member 41 in the circumferential direction of the driveline 2. The sealing member 43 is a member arranged between the inner peripheral surface of the screw member 42 and the outer surface of the driveline 2. The sealing member 43 has, in the holding structure 4, a role of holding the driveline 2 by tightening the driveline 2 and a role of achieving the liquidtightness. It suffices that a material for the sealing member is a material capable of having the role of holding the insertion target through the sealing member by tightening the insertion target and the role of achieving the liquidtightness and, as a representative thereof, an elastic material may be used. For example, the material for the sealing member 43 is preferably an elastic resin material, which elastic resin material includes a single synthetic resin composition such as an elastic silicone resin, and a mixture of a synthetic resin and an inorganic compound.

The inserting member-side pressing portion 43a is a member to keep the liquidtightness by contacting and elastically pressing the outer surface of the driveline 2. In the embodiment, the inserting member-side pressing portion 43a is configured with a surface being parallel to the outer surface of the driveline 2.

The sealing member fitting portion 43b is a portion to contact the inner surface of the screw member 42. The sealing member fitting portion 43b is a portion to contact the screw member fitting portion 42e of the screw member 42. The sealing member fitting portion 43b has a contact surface being parallel to the pressing surface Sa formed on the first opening 321 side, a contact surface being parallel to the contact surface Sb of the screw member fitting portion 42e, and a contact surface provided such that the inner diameter thereof is reduced toward the second opening 322 side from the first opening 321 side.

The chuck member-side contact surface 43c is a portion to contact the chuck member 41. The chuck member-side contact surface 43c is a portion to contact the sealing member-side contacting portion 41d, and is configured with an inclined surface opposing a part of an inclined surface formed on the first opening 321 side of the chuck member 41 and a surface extended parallel to the radial direction of the communicating portion 32. The chuck member-side contact surface 43c is formed continuously with the sealing member fitting portion 43b. While the chuck member-side contact surface 43c is configured with the inclined surface opposing the part of the inclined surface formed on the first opening 321 side of the chuck member 41 and the surface extended parallel to the radial direction of the communicating portion 32, the chuck member-side contact surface 43c is not limited thereto. It suffices that the chuck member-side contact surface 43c has a shape capable of transmitting a force from the sealing member 43 in a direction in which the chuck member 41 is pushed toward the second opening 322 by contacting at least a part of the chuck member-side contact surface 43c with the chuck member 41.

<Assembling of Holding Structure of Driveline>

Next, an assembling method of the holding mechanism assembly 1 will be described using FIGS. 1 to 3.

The driveline 2 is inserted through the communicating portion 32 of the fixing device 3. For example, the driveline 2 extending to the outside of the living body from a hole provided in the skin S is inserted through the communicating portion 32 of the fixing device 3 by inserting the driveline 2 from the second opening portion 322 of the fixing device 3 to the first opening 321 side.

Moreover, the chuck member 41 is arranged on the outer periphery of the driveline 2. Specifically, the fitting portion 41c of the second chuck member 412 is contacted with the outer periphery of the driveline 2 while contacting the fitting portion 41c of the first chuck member 411 configuring the chuck member 41 is contacted with the outer periphery of the driveline 2. Joining the joining surface 411a of the first chuck member 411 and the joining surface 412a of the second chuck member 412 causes the chuck member 41 to be arranged on the outer periphery of the driveline 2.

The chuck member 41 is inserted into the communicating portion 32 from the first opening 321 side. While the fixing device-side contacting portion 41a of the chuck member 41 inserted into the communicating portion 32 will contact the communicating portion inclined surface 323a of the communicating portion 32, it does not necessarily have to contact the communicating portion inclined surface 323a of the communicating portion 32 at this time. Next, the sealing member 43 is fitted to the outer periphery of the driveline 2 such that the sealing member 43 is on the upper side (the first opening 321 side) of the chuck member 41. Thereafter, the screw member 42 to which the O-ring 5 is fitted is fitted to the outer periphery of the driveline 2 such that the screw member 42 is on the upper side (the first opening 321 side) of the sealing member 43. Then, the male screw portion 42c of the screw member 42 is screwed into the female screw portion 323b of the communicating portion 32. The screw member 42 moves toward the second opening 322 side of the fixing device 3 by screwing the male screw portion 42c into the female screw portion 323b.

By screwing the male screw portion 42c of the screw member 42 into the female screw portion 323b of the communicating portion 32, the tip contacting portion 42a of the screw member 42 moves toward the second opening 322, and the tip contacting portion 42a presses the screw member-side contacting portion 41b of the chuck member 41. The chuck member-side contact surface 43c of the sealing member 43 presses the sealing member-side contacting portion 41d of the chuck member 41. At that time, in a case that the chuck member 41 has not been contacted with the communicating portion inclined surface 323a of the communicating portion 32, the chuck member 41 moves toward the second opening 322 side and the fixing device-side contacting portion 41a of the chuck member 41 contacts the communicating portion inclined surface 323a of the communicating portion 32. Then, as screwing of the male screw portion 42c into the female screw portion 323b proceeds, the movement of the chuck member 41 is regulated so that a force to bring the driveline 2 into close contact with the fixing device 3 to hold the fixing device 3 is generated. This makes it possible to assemble the holding mechanism assembly 1 comprising the holding structure 4 having a high close contactability.

The above-described assembling procedure is one example, so that the assembling procedure up to causing the male screw portion 42c of the screw member 42 to be screwed into the female screw portion 323b of the communicating portion 32 is not particularly limited.

The structure of the above-described holding mechanism assembly 1 is merely one example, so that the structure of the holding mechanism assembly is not limited to the embodiment.

At the time attaching of the holding mechanism assembly 1 to the living body is completed, as shown in FIG. 5, a space SP is formed, on the second opening 322 side of the communication path 323 with respect to the holding structure 4, between the inner wall of the communicating portion 32, which defines the communication path 323, and an outer surface 2a of the driveline 2. The space SP is a space formed outward of the outer surface 2a of the driveline 2 in the radial direction between an end E on the second opening 322 side of the holding structure 4 and the opening edge of the second opening 322. When a predetermined time period has passed after the holding mechanism assembly 1 being attached to the living body, the space SP is changed from a state such that cells have not reproduced in the space SP (a state shown in FIG. 5) to a state as shown in FIG. 1, in which the cells has reproduced to cause the space SP to be filled up with the reproduced cells.

As a result of intensive studies, the inventor has found that inflammation occurring in the site at which the fixing device 3 is fixed to the skin S, the cause of which inflammation was not known conventionally, occurs in a case that the driveline 2 is covered with fiber at a position corresponding to the space SP. That is, the inventor has found that the cause of the damage of the cells is a force being applied in the direction in which cells entering into the fiber and cells in the space SP are peeled off due to a relative movement of the driveline 2 in a case that the driveline 2 is covered with fiber at the position corresponding to the space SP. Then, the embodiment makes it possible to suppress cells being damaged by providing the driveline 2 with a non-covered region A2 (described below) at a predetermined position. Below, this point will be described in detail.

According to the embodiment, as shown in FIGS. 1 and 5, the outer surface 2a of the driveline 2 has a covered region A1 covered with fiber that can guide cells and the non-covered region A2 not covered with fiber, and the non-covered region A2 of the driveline 2 extends between the holding structure 4 and the second opening 322 in the communication path 323.

The covered region A1 is a region of the outer surface 2a of the driveline 2, which is covered with fiber that can guide the cells. A material for the fiber constituting the covered region A1 is not particularly limited as long as the cells can be guided and the material is not harmful to the living body. For example, a polyester fiber can be used for the covered region A1. The structure of the covered region A1 constituted by fiber is not particularly limited as long as the cells can be guided. It is preferable that the covered region A1 is formed in a porous shape, for example. The growth of cells is promoted in the covered region A1 by the covered region A1 formed in a porous shape causing cells to enter into a plurality of air gaps formed in the covered region A1.

The non-covered region A2 is a region of the outer surface 2a of the driveline 2, in which the fiber that can guide the cells is not provided. As shown in FIG. 5, the non-covered region A2 is provided at a position of the communication path 323 between the holding structure 4 and the second opening 322, or in other words opposes the space SP in the radial direction of the driveline 2. Since the fiber is not provided in the non-covered region A2, it is suppressed that cells growing into the space SP are fixed to the outer surface 2a of the driveline 2 as described below. Here, the cells being “fixed” refers to a state in which, as in the fiber of the covered region A1, for example, the cells grow inwardly in the radial direction of the driveline 2 to enter into pores formed in the fiber and the cells are fixed to the driveline 2. In the embodiment, the non-covered region A2 has a surface such that damaging of cells invading into the space SP is suppressed when the fixing device 3 moves relatively in the axial direction and/or the radial direction of the driveline 2 with respect to the driveline 2.

Next, functions and effects of the holding mechanism assembly 1 in the embodiment will be described. In the embodiment, as shown in FIGS. 1 and 5, the non-covered region A2 of the driveline 2 extends between the holding structure 4 and the second opening 322. This makes it possible to suppress the cells being damaged in the space SP formed between the outer surface 2a of the driveline 2 and the inner wall of the communicating portion 32. Below, functions and effects of the embodiment will be described in further detail, along with a reference example of a case in which the non-covered region A2 is not provided.

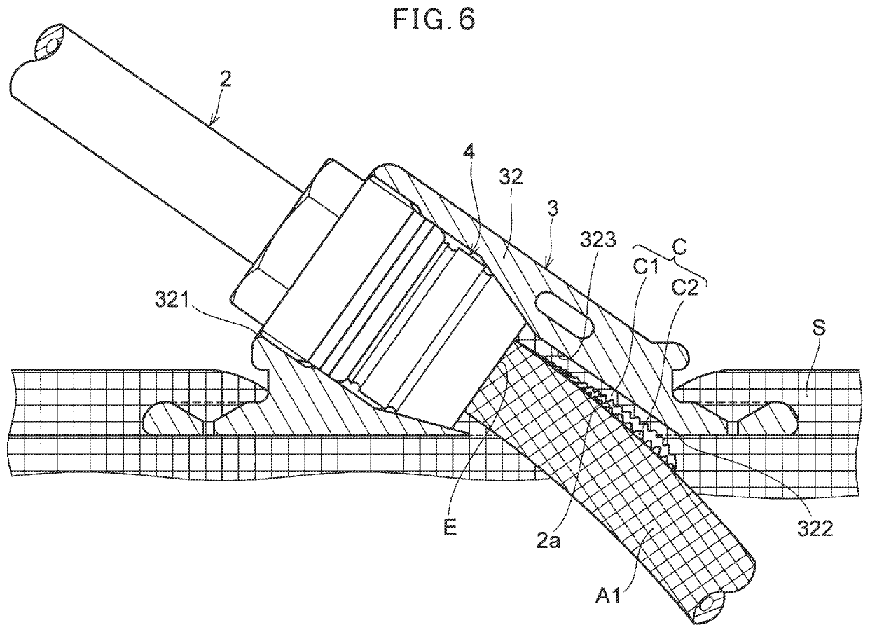

As shown in FIG. 6, in case that the covered region A1 has the fiber provided at a position opposing the space SP, cells C invading into the space SP (see FIG. 5) between the outer surface 2a of the driveline 2 and the inner wall of the communicating portion 32 grow inwardly in the radial direction of the driveline 2 toward pores, which are formed with the fiber, of the covered region A1. Thus, the cells C enter into complex and intricate pores of the covered region A1 to be firmly fixed to the driveline 2. In this state, if the living body performs an action and the fixing device 3 moves with respect to the skin S, as shown in FIG. 6, for example, the driveline 2 and the fixing device 3 move relatively with respect to each other such that the distance between the outer surface 2a of the driveline 2 and the inner wall of the communicating portion 32 increases. Alternatively, the cells in the space SP and the outer surface 2a of the driveline 2 can move relatively in the axial direction of the driveline 2 when the driveline 2 is pulled in the axial direction of the driveline 2, for example. In this way, in case that the outer surface of the driveline 2 moves relatively in the axial direction and/or radial direction of the driveline 2 with respect to the cells C in the space SP, as shown in FIG. 6, cells C2 entering into the fiber in the covered region A1 are peeled off in the radial direction of the driveline 2 or peeled off in the axial direction of the driveline 2 from cells C1 positioned in the space SP.

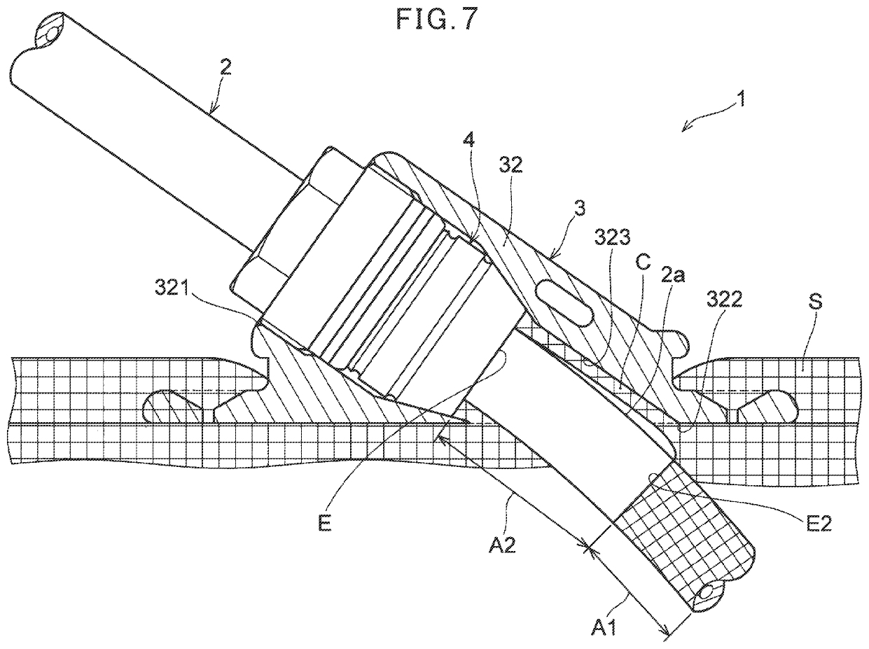

On the other hand, in the embodiment, as shown in FIGS. 1 and 5, the non-contact region A2 not covered with fiber extends between the holding structure 4 and the second opening 322. Thus, as shown in FIG. 7, since intricate pores formed with fiber is not present, the cells C invading into the space SP is not fixed to the outer surface 2a of the driveline 2. Therefore, as shown in FIG. 7, even when the driveline 2 and the fixing device 3 move relatively with each other such that the distance between the outer surface 2a of the driveline 2 and the inner wall of the communicating portion 32 increases, the cells invading into the space SP are easily separated from the outer surface 2a of the driveline 2 so that damage of the cells C is suppressed. Similarly, also when the driveline 2 moves relatively in the axial direction of the driveline 2 with respect to the cells in the space SP, the cells C are easily separated from the outer surface 2a of the driveline 2 so that damage of the cells C is suppressed. Therefore, the holding mechanism assembly 1 according to the embodiment makes it possible to suppress the cells being damaged in the space SP formed between the outer surface 2a of the driveline 2 and the inner wall of the communicating portion 32.

The structure of the non-covered region A2 is not particularly limited as long as the non-covered region A2 is not covered with fiber and the grown cells in the space SP being fixed to the driveline 2 is suppressed. The non-covered region A2 is preferably formed with a smooth curved surface. In case that the non-covered region A2 is formed with the smooth curved surface, it is suppressed that the cells invading into the space SP is fixed to the non-covered region A2. Thus, if a relative movement between the outer surface 2a of the driveline 2 and the cells occurs, the outer surface 2a of the driveline 2 and the cells invading into the space SP are easily separated from each other so that damage of cells is further suppressed. As long as the outer surface 2a of the driveline 2 is smooth, the non-covered region A2 may be in a state in which the tube body of the driveline 2 has no coating, that is, a state in which the material itself of the tube body of the driveline 2 is exposed to the surface, or the tube body of the driveline 2 has a coating with a material other than fiber. A material forming the non-covered region A2 is not particularly limited as long as it is a biocompatible material. The material of the non-covered region A2 may be a synthetic resin, for example.

In the embodiment, the driveline 2 inserted into the fixing device 3 is configured such that the covered region A1 is arranged in a predetermined region at a side toward the inside of the living body with respect to the second opening 322 as shown in FIGS. 1, 5 and 7. In this way, while suppressing the cells being damaged in the space SP, the cells are fixed to the covered region A1 of the outer surface 2a of the driveline 2 in a position at a side toward the inside of the living body in the vicinity of the second opening 322 of the fixing device 3. The holding mechanism assembly 1 has the holding structure 4 in which the fixing device 3 firmly holds the inserting member 2, the non-covered region A2 may be provided between the holding structure 4 and the covered region A1. The holding mechanism assembly 1 having the covered region A1 allows stabilizing the driveline 2 inside the living body to promote healing of the incised site. In a region at a side toward the living body with respect to the second opening 322 of the fixing device 3, influence of the relative movement of cells with respect to the driveline 2 of the fixing device 3 is smaller compared to the surface side of the skin S. The predetermined region of the driveline 2 at a side toward the living body with respect to the second opening 322 is not particularly limited as long as the predetermined region is in the vicinity of the second opening 322 and cells can be fixed in the covered region A1 of the driveline 2. For example, in the covered region A1, it is preferable that a depth D at the deepest portion of an end (a border between the covered region A1 and the non-covered region A2) E2 of the covered region A1 in FIG. 1 from the opening edge of the second opening 322 is set to be less than or equal to 7-10 mm (or D≤10 to 15 mm). In this case, it is possible to grow cells across the entire outer periphery of the fiber of the covered region A1 of the driveline 2 to promote healing of the incised site while suppressing the cells being damaged. In the embodiment, the covered region A1 is provided such that the end E2 of the covered region A1 extends in a direction being perpendicular to the axial direction of the driveline 2. However, as shown with a double-dashed line E20 in FIG. 5, the end of the covered region A1 may extend so as to be parallel to the opening edge of the second opening 322 of the fixing device 3.

### EXPLANATIONS OF LETTERS

1 HOLDING MECHANISM ASSEMBLY
2 DRIVELINE (INSERTING MEMBER)
2a OUTER SURFACE OF DRIVELINE
21 COOLING WATER CIRCULATION PATH
22 POWER CABLE
3 FIXING DEVICE
31 FIXING PORTION
31a POROUS PORTION
31b FLAT PORTION
32 COMMUNICATING PORTION
321 FIRST OPENING
322 SECOND OPENING
323 COMMUNICATION PATH
323a COMMUNICATING PORTION INCLINED SURFACE
323b FEMALE SCREW PORTION
33 COUNTERSINK PORTION
4 HOLDING STRUCTURE
41 CHUCK MEMBER
41a FIXING DEVICE-SIDE CONTACTING PORTION
41b SCREW MEMBER-SIDE CONTACTING PORTION
41c FITTING PORTION
41d SEALING MEMBER-SIDE CONTACTING PORTION
41e CONCAVE PORTION
411 FIRST CHUCK MEMBER
411a JOINING SURFACE
412 SECOND CHUCK MEMBER
412a JOINING SURFACE
42 SCREW MEMBER
42a TIP CONTACTING PORTION
42b SCREW MEMBER INSERTING PATH
42c MALE SCREW PORTION
42d TIP PORTION
42e SCREW MEMBER FITTING PORTION
42f RECEIVING PORTION
42g CONCAVE PORTION
43 SEALING MEMBER
43a INSERTING MEMBER-SIDE PRESSING PORTION
43b SEALING MEMBER FITTING PORTION
43c CHUCK MEMBER-SIDE CONTACT SURFACE
5 O RING
A1 COVERED REGION
A2 NON-COVERED REGION
C, C1, C2 CELLS
E END ON SECOND OPENING SIDE OF HOLDING STRUCTURE
E2 END OF COVERED REGION
S SKIN
Sa PRESSING SURFACE
Sb CONTACT SURFACE
Sc CONTACT SURFACE
SP SPACE

1. A holding mechanism assembly comprising:
a tubular inserting member, one end side of which is to be arranged in an inside of a living body and the other end side of which is to be arranged in an outside of the living body;
a fixing device having a fixing portion to be fixed beneath a skin of the living body and a communicating portion into which the inserting member is inserted; and
a holding structure provided in the communicating portion to hold the inserting member to the fixing device in a liquidtight manner, wherein
the communicating portion has a first opening opened toward the outside of the living body when the fixing device is fixed beneath the skin; a second opening opened toward the inside of the living body when the fixing device is fixed beneath the skin; and a communication path formed between the first opening and the second opening;
a space is formed, on the second opening side of the communication path with respect to the holding structure, between an inner wall of the communicating portion, which defines the communication path, and an outer surface of the inserting member;
the outer surface of the inserting member has a covered region covered with fiber that can guide cells and a non-covered region not covered with the fiber; and
the non-covered region of the inserting member extends between the holding structure and the second opening in the communication path.

2. The holding mechanism assembly according to claim 1, wherein the non-covered region is formed with a smooth curved surface.

3. The holding mechanism assembly according to claim 1, wherein the inserting member inserted into the fixing device is configured such that the covered region is arranged in a predetermined region at a side toward the inside of the living body with respect to the second opening.

4. The holding mechanism assembly according to claim 1, wherein the covered region is formed in a porous shape.
